(19) United States
(12) Patent Application Publication
(10) Pub. No.: US 2025/0361450 A1
(43) Pub. Date: Nov. 27, 2025

(54) Producing Synthetic Fuels from Acid Gas Streams

(71) Applicant: Saudi Arabian Oil Company, Dhahran (SA)

(72) Inventors: Mohammad Saad AlQahtani, Dhahran (SA); Ahmed W. Ameen, Dhahran (SA); Melhan M. Ben Sultan, Dammam (SA); John P. O'Connell, Dhahran (SA)

(21) Appl. No.: 18/671,258

(22) Filed: May 22, 2024

(51) Int. Cl. C10G 2/00; C07C 29/152; C07C 29/88; C10G 45/04

(52) U.S. Cl. CPC C10G 2/34; C07C 29/152; C07C 29/88; C10G 2/35; C10G 45/04; C10G 2300/202; C10G 2300/207; C10G 2300/4081

(57) ABSTRACT

A system and method for producing methanol and synthetic fuels from waste acid gas streams using a plasma reactor is described in this disclosure. An acid gas stream comprising primarily of H₂S and CO₂ is fed into a plasma reactor. H₂S is converted into H₂ and sulfur. Simultaneously, CO is formed by the reverse water gas shift reaction. H₂ and CO form a syngas stream. The unreacted H₂S is captured in a tail gas treatment unit and recycled back to the plasma reactor. A partial CO₂ capture unit is placed downstream of the tail gas treatment unit which is primarily used to adjust the ratio of H₂ and CO in the syngas stream to 2-3 for methanol production and 2 for fuel production.

### TECHNICAL FIELD

This disclosure relates to a method of producing methanol and synthetic fuels from acid gas streams.

### BACKGROUND

Hydrogen sulfide (H₂S) and carbon dioxide (CO₂) often co-exist in many gas streams, including natural gas. Typically, H₂S and CO₂ are selectively removed from gas mixtures using liquid amine absorption. The concentrated H₂S/CO₂ mixture is fed to the Claus process where only elemental sulfur is recovered. In the Claus unit, hydrogen (H₂) in H₂S is oxidized to water (H₂O) in the reaction furnace. On the other hand, CO₂ ends up in the tail gas and it is emitted to the atmosphere which contributes to a large carbon footprint.

### SUMMARY

This disclosure relates to a process of using acid gas streams as a feed stock for synthetic methanol and/or fuels production. An acid gas stream primarily includes H₂S and CO₂, and varying amounts of water vapor, nitrogen (N₂), hydrocarbons, ammonia (NH₃), and other contaminants. As aspect of this technology uses a plasma reactor to produce H₂ from the H₂S gas and CO from the CO₂ gas stream to produce a syngas stream. The sulfur containing gases from the plasma reactor are treated in a tail gas treatment unit and the unreacted H₂S is recycled to the plasma reactor. The outlet gases from the tail gas treatment unit are sent to a partial CO₂ capture unit. The partial CO₂ capture unit uses an amine-based process or a membrane-based process to enrich the H₂ in the syngas stream, where a ratio of H₂/COₓ of 2-3 is used for methanol synthesis and a H₂/COₓ ratio of 2 is used for synthetic fuel production by the Fisher Tropsch process.

This technology generates value by the production of synthetic fuels from waste acid gases such as H₂S and CO₂ mixture.

### BRIEF DESCRIPTION OF DRAWINGS

FIG. 1 is a block flow diagram for a plasma-based process for synthetic methanol or fuels production and a partial CO₂ capture unit where the partial CO₂ capture unit is placed downstream of the tail gas treatment unit.

FIG. 2 is a block flow diagram for a plasma-based process for synthetic methanol or fuels production and a partial CO₂ capture unit where the partial CO₂ capture unit is placed upstream of the plasma unit.

FIG. 3 is a block flow diagram for a plasma-based process for synthetic methanol or fuels production with enhanced CO concentration.

FIG. 4 is a schematic representation of a plasma unit.

FIG. 5 is a block flow diagram of a tail gas treatment unit.

FIG. 6 is a block diagram for an amine-based partial CO₂ capture unit process.

FIG. 7 is a block diagram for a membrane-based partial CO₂ capture unit process.

FIG. 8A is a block diagram for fuel production via methanol route.

FIG. 8B is a block diagram for fuel production via Fischer-Tropsch route.

FIG. 9 shows the results from a lab scale experiment that demonstrates the formation of H₂ and CO in a non-thermal plasma reactor.

FIG. 10A is an Aspen HYSYS simulation of the conversion of acid gas stream to syngas-50% H₂S in the feed acid gas.

FIG. 10B is an Aspen HYSYS simulation of the conversion of acid gas stream to syngas-75% H₂S in the feed acid gas.

### DETAILED DESCRIPTION

This disclosure relates to a method of producing synthetic methanol and/or synthetic fuels from an acid gas stream. An acid gas stream primarily includes hydrogen sulfide (H₂S) and carbon dioxide (CO₂) gases and varying amounts of water vapor, nitrogen (N₂), hydrocarbons, ammonia (NH₃), and other contaminants. An aspect of this technology relates to using a plasma reactor to split H₂S into H₂ and sulfur, and simultaneously produce CO from CO₂ via reverse water gas shift reaction (RWGS). Methanol and other synthetic fuels are produced via the syngas (H₂/CO) intermediate route.

An implementation described herein provides a method to treat the remaining gases from the plasma reactor in a tail gas treatment unit. The unreacted H₂S in a tail gas treatment unit is recycled back to the plasma reactor. The outlet gases from the tail gas treatment unit are sent to a partial CO₂ capture unit. The partial CO₂ unit is used to enrich the H₂ to COₓ to reach a H₂/COₓ ratio of 2 to 3 for methanol production and a H₂/CO ratio of 2 for synthetic fuel production. In some implementations, a slip stream containing CO₂ from the partial CO₂ capture unit is recycled back to the plasma reactor to enrich the CO formation. The produced syngas (H₂ and CO mixture) is further processed in a methanol synthesis unit to produce methanol or sent to a Fischer Tropsch plant for synthetic fuel production.

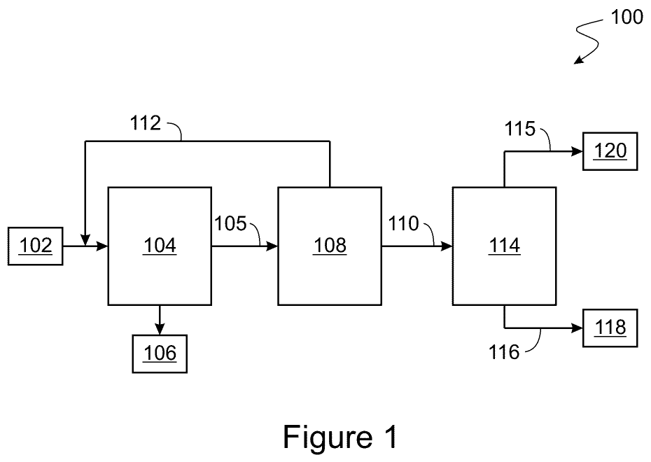

FIG. 1 is a block flow diagram for a plasma-based process 100 for synthetic methanol or fuels production and a partial CO₂ capture unit where the partial CO₂ capture unit is placed downstream of the tail gas treatment unit. An acid gas stream 102 is received by a plasma unit 104. The acid gas stream 102 primarily comprises of H₂S and CO₂ and can also include other contaminant gases such as hydrocarbons, nitrogen (N₂), BTEX (Benzene, Toluene, Xylenes), and ammonia (NH₃) that are typical low (<1%).

The plasma unit 104 includes one or multiple stages of plasma reactors with sulfur condensers between stages to achieve the targeted sulfur recovery level. The plasma reactor is used to simultaneously split H₂S to produce H₂ and elemental sulfur (R1) and to facilitate reverse water gas shift reaction (RWGS) to produce CO (R2). The produced sulfur is recovered as liquid sulfur 106 in a condenser. The reactions proceed as follows:

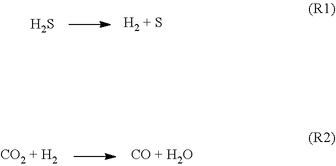

In some implementations, the plasma reactor can be packed with a solid catalyst (plasma catalysis) to increase the rate of the reaction and/or control the selectivity of H₂, CO, and elemental sulfur(S). If significant amount of SO₂ is formed in the plasma reactor, then a catalytic reactor is used downstream the plasma reactor to remove SO₂ via Claus reaction (R3). The reaction proceeds as follows:

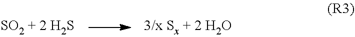

Alumina and/or titania-based catalysts can be used for the Claus reaction. The H₂S to SO₂ ratio being much higher than the stoichiometry for the Claus reaction (H₂S/SO₂>2), allows for complete SO₂ removal. If a small amount of SO₂ formation is anticipated, then alumina and/or titania is packed in the bottom section of the plasma catalytic reactor, which will eliminate the need for a separate catalytic convertor.

The remaining gas 105 from the plasma unit 104 primarily includes H₂, H₂S, CO, COS, CS₂, SO₂, S, CO₂. It is sent to the tail gas treatment unit 108 to recover unreacted H₂S and recycle it back to the plasma unit 104. A typical tail gas treatment unit 108 comprises of a catalytic reactor containing hydrogenation/hydrolysis catalyst(s) to convert all sulfur-containing gases (e.g., SO₂, COS, S, etc.) to H₂S (R4-R6). The reactions proceed as follows:

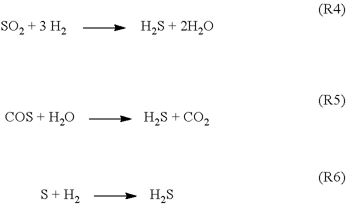

The excess water formed during the reactions R4-R6 is removed in a quenching tower. The gases leaving the top of the quenching tower is mainly a mixture of H₂S, H₂, CO, and CO₂. The H₂S/CO₂ gas mixture 112 is selectively removed by liquid amine absorption and recycled back to the plasma unit 104. If the ratio of H₂ to COₓ falls below 2.2 for methanol production or below 2 for fuels production, then the remaining H₂, CO, and CO₂ gas stream 110 is sent to a partial CO₂ capture unit 114 (e.g., liquid amine absorption, CO₂ selective membrane, pressure swing adsorption, etc.) to increase the H₂ to COₓ ratio by partial CO₂ removal. Finally, the syngas stream (H₂/COₓ) 115 is sent to a methanol or Fischer-Tropsch plant 120.

The sulfur-free syngas stream 115 produced from acid gas streams is sent to an existing methanol plant for synthetic methanol production and/or synthetic fuels (such as jet, diesel, gasoline, etc.) via methanol to olefin route. Alternatively, syngas stream 115 can be sent to a typical Fischer-Tropsch process for fuels production. The electricity needed for the plasma reactor to produce the syngas can be obtained from sustainable renewable electricity sources such as wind, solar, etc.

In some implementations in process 100, a partial CO₂ capture unit 114 can be an amine-based process. In other implementations, it can be based on a membrane process where a CO₂ selective membrane is deployed to concentrate CO₂ and produce a pure CO₂ stream 116 in the permeate stream, that may be sent for a second stage to recover the slipped H₂. The advantage of using a membrane-based process is that the syngas will be obtained at a high pressure and can be sent directly to methanol or Fischer-Tropsch plants without additional compression.

The plasma reactor can use different types of plasma such as dielectric barrier discharge (DBD), corona discharge, pulsed corona discharge, spark, glow, gliding arc, thermal arc, and microwave plasmas. These plasmas differ significantly in terms of their properties and the way they are generated. For example, temperatures could be as low as room temperature (non-thermal plasma) or thousands of degrees (thermal plasma).

Thermal plasma is limited by thermodynamic equilibrium. Thus, an extremely fast quenching is required after the plasma reaction to prevent recombination of sulfur and H₂. However, nonthermal plasma (NTP) is a non-equilibrium process. At low temperatures it contains radicals and excited states of atoms and molecules that exist at thermal equilibrium only at much higher temperatures (>1000° C.). The chemical processes occurring for NTP are not possible in a system that is at thermal equilibrium.

In nonthermal plasma, highly energetic electrons interact with gas molecules (electron impact reactions) to produce radicals, ions, and rotationally, vibrationally, and/or electronically excited molecules that facilitate chemical reactions at mild conditions. The non-equilibrium nature of NTP allows high H₂S conversion to take place at low temperatures. A common example of NTP is DBD where plasma is generated when the voltage between two electrodes (at least one of which is covered by a layer of dielectric material) is higher than the breakdown voltage of the gas passing in between the two electrodes. The minimum voltage difference required to generate NTP depends on the gas composition, pressure, and the distance between the two electrodes. Non-thermal plasma can be operated at a wide range of temperatures such as 30-900° C. and near atmospheric pressure (1-5 bar). Plasma high voltage can range from 1-50 kV with a frequency ranging from lower radio frequency (RF) to microwave frequencies. In certain implementations herein, the process is operated between 15° and 250° C. to minimize sulfur deposition on the catalyst. However, temperatures ranging from 30 to 800° C. can be used.

Catalyst(s) can be coupled with the plasma to increase H₂S conversion, CO₂ conversion, and/or H₂ and CO yield. Plasma can activate catalyst(s) at low temperatures to increase the rate of reactions. A single catalyst, bifunctional catalyst, and/or physical mixture of different catalysts can be utilized to simultaneously catalyze different catalytic reactions, such as H₂S splitting and/or reverse water gas shift reaction. Examples of catalysts are metal sulfide, supported metal sulfide, metal nitrate, supported metal nitrides, zeolite, and carbon-based catalysts.

The concentration of acid gas stream contaminations such as hydrocarbons, BTEX (Benzene, Toluene, Xylenes), and ammonia (NH₃) are typical low (<1%). Plasma can decompose/crack some of these contaminations. For example, NH₃ can decompose to produce H₂ and N₂ via reaction (R7), whereas hydrocarbons can be cracked to lighter hydrocarbons (R8-R9). The reaction proceeds as follows:

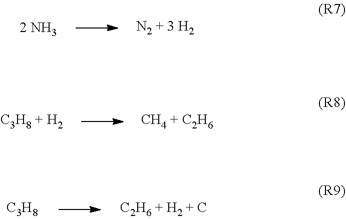

Water vapor also exists in the acid gas stream (˜5%). The effect of these contaminants on the quality of H₂ and CO₂ is minimal due to their low concentration.

In some implementations, the partial CO₂ unit can be placed upstream of the plasma unit.

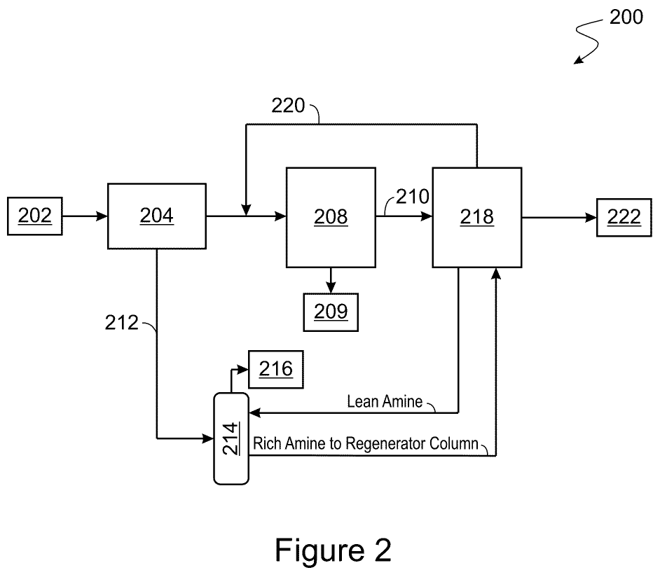

FIG. 2 is a block flow diagram for a plasma-based process 200 for synthetic methanol or fuels production and a partial CO₂ capture unit, where the partial CO₂ capture unit is placed upstream of the plasma unit. An acid gas stream 202 that primarily includes H₂S and CO₂ is sent to a partial CO₂ capture unit 204. The partial CO₂ capture unit 204 includes a CO₂ selective membrane, where the membrane is used to concentrate CO₂ to form a CO₂ rich stream 212 and send it to amine absorber column 214 to capture the slipped H₂S gas. The H₂S gas is then sent to a tail gas treatment unit 218. This configuration requires the addition of a membrane and only one amine absorber column 214, as it utilizes the tail gas treatment unit 218 regenerator column to regenerate the rich amine. The lean amine is further processed in the absorber column 214. Pure CO₂ stream 216 is obtained from the amine absorber column 214.

The reject gas from the partial CO₂ unit 204 is sent to the plasma unit 208. The reject gas contains a higher ratio of H₂S to CO₂. The plasma unit 208 includes a single stage or multi-stage plasma reactor with sulfur condensers between stages. The H₂S gas splits into H₂ and sulfur in the plasma reactor. The produced sulfur is recovered as liquid sulfur 209 in a condenser. A reverse water gas shift reaction produces CO from the CO₂. The resulting gas stream 210 along with other contaminant gases or by-products of the plasma induced reaction are sent to the tail gas treatment unit 218. The resulting gas stream 210 primarily comprises of H₂, H₂S, CO, CO₂ and a very small fraction of contaminant gases. The contaminant gases do not affect the H₂ or COₓ gas streams formed.

The tail gas treatment unit 218 comprises of a catalytic reactor containing hydrogenation/hydrolysis catalyst(s) to convert all sulfur-containing gases (e.g., SO₂, COS, S, etc.) to H₂S (reactions R4-R6). The excess water formed is removed in a quenching tower. The gas leaving the top of the quenching tower is mainly a mixture of H₂S, H₂, CO, and CO₂. The H₂S is selectively removed by liquid amine absorption and recycled back as H₂S/CO₂ recycle stream 220 to the plasma unit 208. The produced syngas stream (H₂ and COₓ) 222 is processed by a methanol plant or a Fischer-Tropsch plant to produce synthetic fuels. The ratio of H₂ and CO is maintained at 3 for methanol production and at 2 for synthetic fuel production. In a typical methanol plant, up to 10% CO₂ can be co-fed with syngas, hence, the need for only partial CO₂ removal which requires less CAPEX and OPEX compared to complete CO₂ removal.

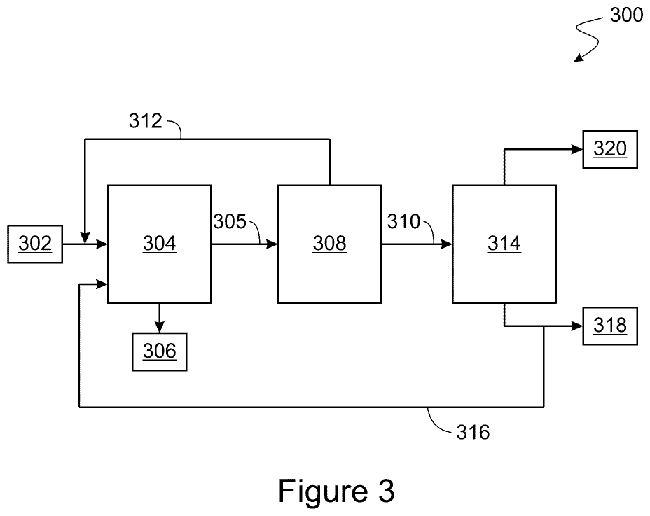

FIG. 3 is a drawing of a block flow diagram for plasma-based process for synthetic methanol or fuels production with enhanced CO concentration.

In certain implementations, a higher concentration of CO is desired for syngas production. An acid gas stream 302 primarily comprising of H₂S and CO₂ is sent to a plasma unit 304. The plasma unit 304 includes single stage or multi-stage plasma reactors with sulfur condensers placed between stages. The H₂S from the acid gas stream 302 is split into H₂ and sulfur, where sulfur is recovered by the condenser as liquid sulfur 306. A reverse water gas shift reaction produces CO. The remaining gases 305 which primarily include H₂, H₂S, CO, and CO₂ are sent to the tail gas treatment unit 308.

The tail gas treatment unit 308 comprises of a catalytic reactor containing hydrogenation/hydrolysis catalyst(s) to convert sulfur-containing gases (e.g., SO₂, COS, S, etc.) to H₂S (R4-R6). In some implementations, an optional H₂O removal unit (e.g., molecular sieve) can be installed prior to the catalytic hydrogenation stream to minimize the water gas shift reaction. The unreacted H₂S in the tail gas treatment unit 308 is selectively removed by liquid amine absorption and sent as a recycle stream 312 back to the plasma unit 304. The syngas stream 310 from the tail gas treatment unit 308 is received by the partial CO₂ unit 314. The partial CO₂ unit is used to remove CO₂ and produce a pure CO₂ stream 318 and a syngas stream 320 enriched with H₂ such that the ratio of H₂/COₓ is maintained at 2-3 for methanol production and at 2 for synthetic fuel production. The syngas stream 320 is sent to a methanol or Fischer Tropsch plant. The

If a high CO production is required in the plasma unit 304, a slip stream of pure CO₂ 316 is recycled to the feed of the plasma unit 304.

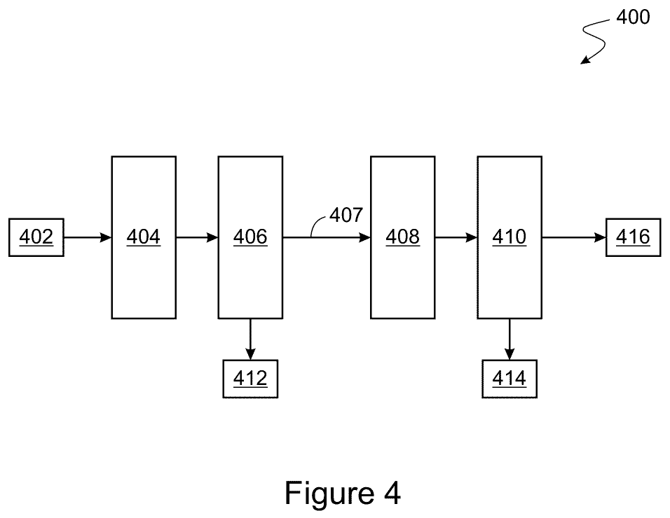

FIG. 4 is a schematic representation of a plasma unit 400. An acid gas stream 402 comprising primarily of H₂S and CO₂ is processed by a plasma reactor 404. The plasma reactor 404 can be packed with a solid catalyst, called as plasma catalysis, to increase the rate of the reaction and/or control the selectivity to H₂, CO, and elemental sulfur. In the event a small quantity of SO₂ is formed, then alumina and/or titania that is packed in the bottom section of the plasma catalytic reactor will be used to eliminate it. The plasma catalyst can include a single catalyst, bifunctional catalyst, or physical mixture of different catalysts are used to simultaneously catalyze different catalytic reactions and/or enhance plasma properties. The catalysts can be, but not limited to, supported or unsupported metal sulfide-based catalysts such as molybdenum or zinc sulfides supported on alumina or silica, carbon- or zeolite-based catalysts.

The plasma reactor 404 includes one of dielectric barrier discharge (DBD), corona discharge, pulsed corona discharge, spark, glow, gliding arc, thermal arc, and microwave discharge plasmas. These plasmas differ significantly in terms of their properties and the way they are generated. For example, temperatures could be as low as room temperature (non-thermal plasma) or thousands of degrees (thermal plasma). Plasma high voltage can range from 1-50 kV with a frequency ranging from lower radio frequency (RF) to microwave frequencies. The operating pressure can range from 1-5 bar, depending on the type of plasma process. In the preferred practices, the process is operated between 15° and 250° C. to minimize sulfur deposition on the catalyst, however, temperature ranging from 30 to 800° C. can be used. The plasma reactor 404 requires only electricity to operate which can be obtained from renewable sources.

A condenser 406 is placed downstream of the plasma reactor 404 to transfer the heat from a vapor phase to liquid phase. The condenser 406 can include a heat exchanger and it can either be air-cooled or water-cooled. The condenser 406 is used to recover elemental sulfur as a liquid 412. The plasma reactor converts H₂S to H₂ and sulfur. The sulfur gas is cooled down in the condenser 406 to a liquid phase and sulfur is recovered as a useful product. In some implementations, significant SO₂ is formed in the plasma reactor 404. In this case, a catalytic reactor 408 is placed downstream of the condenser 406, to remove SO₂ by Claus reaction (R3). The catalytic reactor 408 is filled with alumina and/or titania catalyst. The catalytic convertor 408 receives a gas stream 407 from the condenser 406 which primarily includes H₂, H₂S, SO₂, CO, CO₂. The plasma unit 400 has multiple stages. Another condenser 410 is placed downstream of the catalytic reactor 408 to recover liquid sulfur 414. The gas stream 416 coming out of the condenser 410 is comprised primarily of H₂, H₂S, CO, and CO₂. It is sent to the tail gas treatment unit for further processing, where the unreacted H₂S is removed and recycled back to the plasma unit 400.

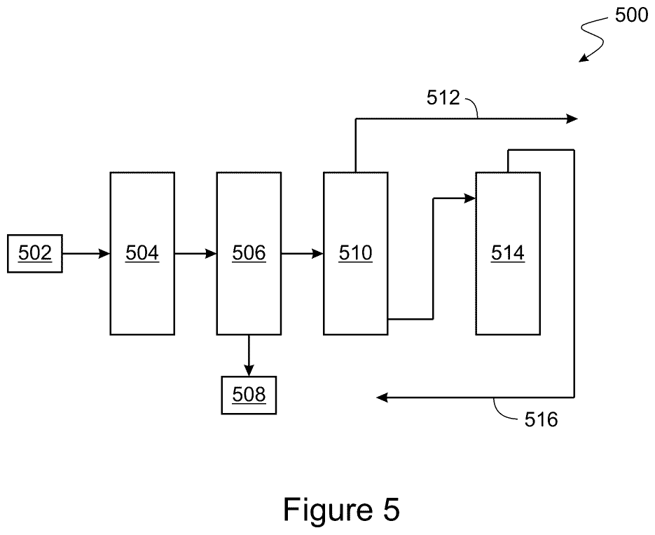

FIG. 5 is a schematic representation of a tail gas treatment unit 500. The gas stream 502 from the plasma unit is sent to the catalytic reactor 504 which contains a hydrogenation or hydrolysis catalyst. This catalyst converts all sulfur-containing gases (e.g., SO₂, COS, S, etc.) to H₂S that proceed via reactions R4-R6. A quenching tower 506 is placed downstream of the catalytic reactor 504. The excess water 508 formed during the reaction is removed by the quenching tower 506. The gases leaving the top of the quenching tower 506 is mainly a mixture of H₂S, H₂, CO, and CO₂. An absorber column 510 is placed downstream of the quenching tower 506. The gases from the quenching tower 506 passes through the absorber column 510, where the gas stream is contacted by a counter-current amine stream. Chemical reactions occur between the acid gases and the amine solution. The amine solution selectively absorbs H₂S and a rich amine stream is formed. The rich amine stream that contains the absorbed gases enters a regenerator column 514 which is installed downstream of the absorber column. The process of removing sulfur containing gases from an acid gas stream is known as ‘sweetening’. The absorber column 510 can include packed beds, spray column, tray columns, bubble columns, or scrubbers. The gas stream 512 leaving the absorber column primarily includes H₂, CO, and CO₂.

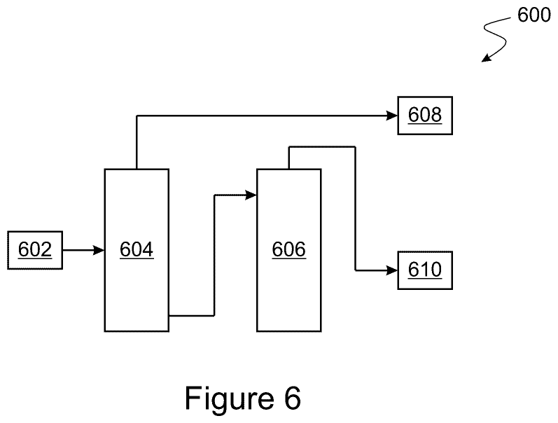

A regenerator column 514 is used to strip the absorbed gases from the amine solution, such that the regenerated amine solution can be recycled as a ‘lean amine stream’ back to the absorber column 510. This is further used to sweeten the incoming acid gas mixtures in the absorber column 510. The recovered H₂S and CO₂ gas stream 516 from the regenerator is recycled back to the plasma unit. FIG. 6 is a block diagram for a partial CO₂ capture unit 600 that is an amine-based process. The gas stream 602 coming from a tail gas treatment unit enters a partial CO₂ capture unit 600. The partial CO₂ capture unit can be an amine-based process or a membrane-based process. The amine-based process includes an amine absorber column 604 as shown in FIG. 6. This amine solution includes monoethanolamine, diethanolamine, di-isopropanolamine, or methyldiethanolamine. The amine solution absorbs CO₂ gas. The treated gas 608 leaves the absorber column 604 at the top. The treated gas 608 is syngas (H₂, CO, CO₂). The ratio of the syngas is adjusted such that H₂/COₓ is 2-3 for methanol production and 2 for synthetic fuel production via Fischer-Tropsch process. In methanol plants, up to 10% CO₂ is co-fed with the syngas, requiring only partial CO₂ removal. This requires systems with less CAPEX and OPEX compared to systems that require complete CO₂ removal. The CO₂ rich amine solution from the absorber column 604 is directed towards a regeneration column 606. In the regeneration column 606, the CO₂ is desorbed and pure CO₂ gas 610 is obtained.

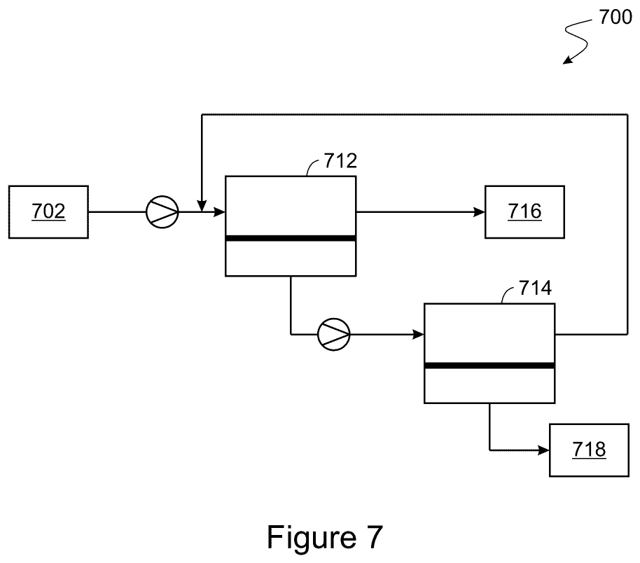

FIG. 7 is a block diagram for a partial CO₂ capture unit 700 that is a membrane-based process. A processed syngas stream 702 from the tail gas treatment unit is sent to a CO₂ selective membrane 712. This membrane produces concentrated CO₂ in the permeate stream. In some implementations, the process uses one or more than one CO₂ selective membrane. In implementations herein, two CO₂ selective membrane 712 and 714 are used in series. These membranes operate on the principle of selective permeation. Each gas component has a specific permeation rate. The permeation rate depends on the rate at which the gas dissolves into the membrane surface and the rate at which it diffuses through the membrane. The second CO₂ selective membrane 714 is mainly used to recover the slipped hydrogen gas. A CO₂-rich permeate stream 718 is obtained from the membrane 714, which can be used for other industrial purposes. The CO₂-lean stream from the membrane 714 is sent for further processing to the first CO₂ selective membrane 712. The reject stream 716 from the first CO₂ selective membrane 712 comprises primarily of H₂, CO, and CO₂. It is processed by the methanol plant or Fischer Tropsch plant. The purpose of the partial CO₂ capture unit is to enrich H₂ in the syngas stream to adjust the ratio to 2-3 for methanol production and to 2 for synthetic fuel production. In some implementations, the CO₂ selective membranes can be used as a single stage process. The advantage of using a membrane process is that the syngas is obtained at a high pressure, and it can be sent directly to methanol or Fischer-Tropsch plant without additional compression.

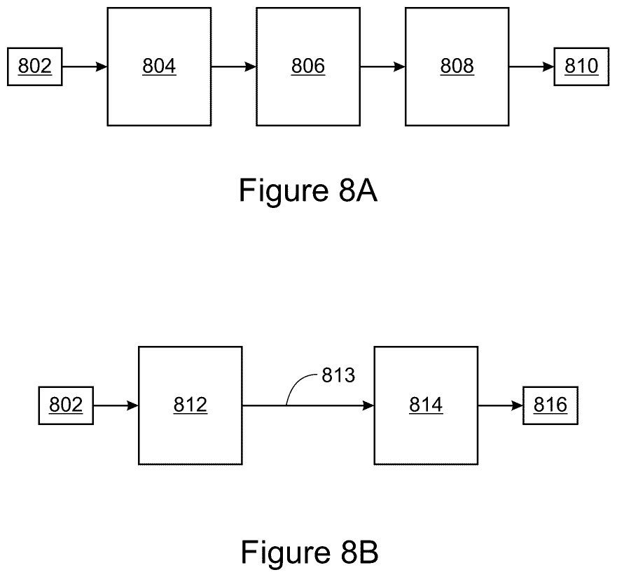

FIG. 8A is a block diagram for fuel production via methanol route. The sulfur free syngas stream 802 produced from an acid gas stream is processed by a methanol plant 804. The methanol plant 804 includes a methanol synthesis reactor to produce methanol from H₂ and CO. The catalyst may include copper, zinc oxide (ZnO), alumina, magnesia, copper oxide (CuO), or aluminum oxide (Al₂O₃), or mixtures thereof. In certain implementations, the catalyst is a mixture of copper and zinc oxides, supported on alumina. The operating temperature may be, for example in a range of 220° C. to 280° C. The methanol synthesis reaction 2H₂+CO→CH₃OH is generally exothermic. Therefore, heat may be removed from the vessel using a heat transfer jacket, a recirculation heat exchanger, or other heat transfer system. In some implementations, unreacted CO, unreacted H₂, and unreacted methanol discharged from the reactor vessel may be recycled to the reactor vessel. The methanol reactor and supporting unit operations can be operated on electricity obtained from renewable sources. The methanol synthesized is processed by a catalytic reactor in the methanol to olefin conversion plant 806. The olefin produced undergoes oligomerization and hydrocracking in a catalytic reactor in the hydrocracking unit 808. This leads to the formation of fuels 810 such as diesel, jet fuels, and gasoline.

FIG. 8B is a block diagram for fuel production via Fischer Tropsch route. The sulfur free syngas stream 802 produced from an acid gas stream is processed by a Fischer Tropsch plant 812. This process synthesizes liquefied hydrocarbons 813. The liquefied hydrocarbons 813 undergo hydrocracking in the hydrocracking unit 814. This leads to the formation of fuels 816 such as diesel, jet fuels, and gasoline.

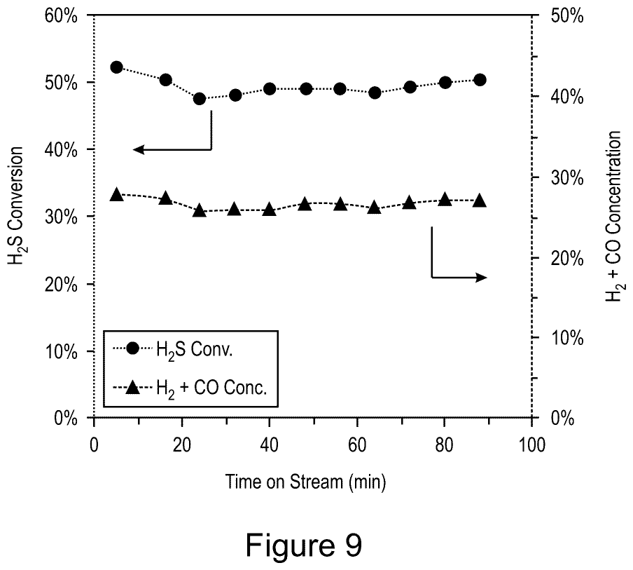

FIG. 9 is a lab scale experiment that demonstrates the conversion of H₂S to H₂ and CO in a non-thermal plasma reactor. A feed gas comprising of 46% H₂S, 53% CO₂, and 1% hydrocarbons was fed into a non-thermal plasma reactor at 150° C. H₂S conversion of around 50% was achieved in a single pass. The residence time was 12s. The produced H₂ and CO concentration in the product stream was around 25%.

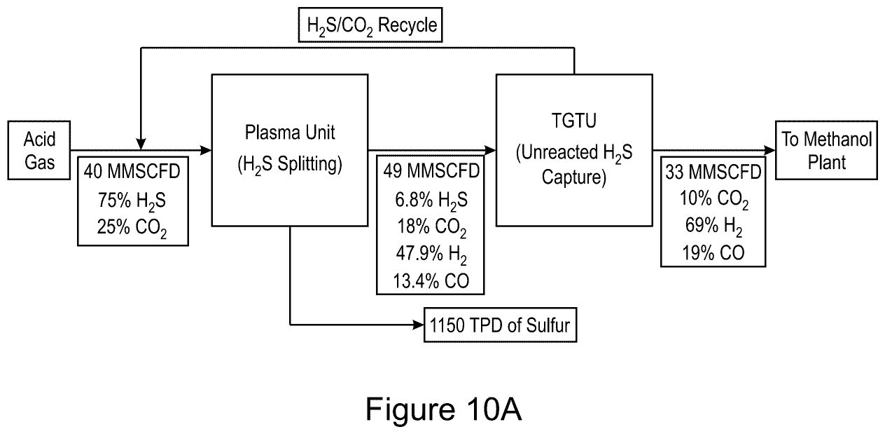

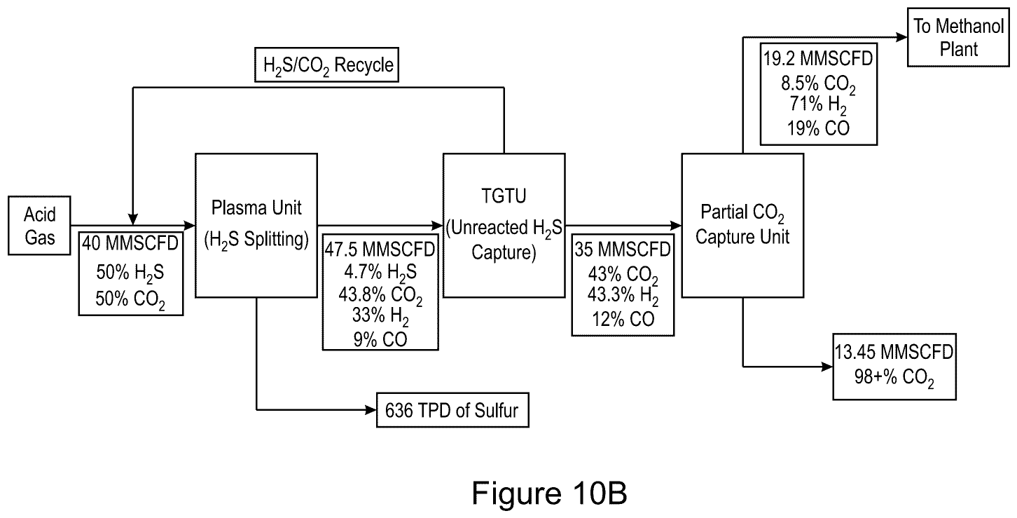

FIG. 10A and FIG. 10B are Aspen HYSYS simulations for the conversion of acid gas stream to syngas. Two different simulations A and B were conducted at different H₂S concentrations in the feed. Simulation A has 50% H₂S in the feed acid gas and simulation B has 75% H₂S in the feed acid gas. In simulation A, the H₂S concentration is 75% which is high enough when compared to the CO₂ concentration. Hence, there is no requirement of using a partial CO₂ capture unit to adjust the H₂ to COₓ ratio in the output syngas stream. In the case of simulation B, the H₂S concentration is less than 75% in the feed acid gas stream. Hence, the need for a partial CO₂ capture unit to adjust the H₂ to COₓ ratio in the output syngas stream. In simulation B where H₂S concentration is 50% in the acid gas stream, around 67% of CO₂ has to be removed/captured to adjust the H₂/COₓ ratio to 3, which is needed for methanol production. A mass balance based on Aspen HYSYS simulation is presented in FIG. 9 for both cases A and B.

An embodiment described herein provides a method to use acid gas streams, primarily a mixture of H₂S and CO₂, as a feedstock for synthetic methanol and/or fuels production. This disclosure uses H₂S as a H₂ source and CO₂ as a carbon source to produce methanol and/or fuels via the syngas (H₂/CO) intermediate route.

An embodiment described here relates to a method for producing methanol and synthetic fuels from an acid gas stream, primarily a mixture of H₂S and CO₂. The acid gas stream is received by a plasma reactor. The plasma reactor produces a syngas stream by simultaneously splitting H₂S into H₂ and sulfur and forming CO by the reverse water gas shift reaction. The sulfur is obtained as a liquid in a sulfur condenser placed downstream of the plasma reactor.

In some implementations, the plasma reactor has multiple stages with at least one sulfur condenser between stages to recover liquid sulfur. The remaining gases are processed by a tail gas treatment unit to capture and recycle unreacted H₂S back to the plasma reactor. The ratio of H₂ to CO in the syngas stream at the outlet of the tail gas treatment unit is adjusted to a value of 2-3 for producing methanol and a value of 2 for producing synthetic fuel. A partial CO₂ removal unit is used to adjust the ratio of H₂ to CO.

An aspect described here relates to a system for producing methanol and synthetic fuels. The system includes a plasma unit, a tail gas treatment unit which is placed downstream of the plasma unit, and a partial CO₂ capture unit installed downstream of the tail gas treatment unit.

The plasma unit includes a non-thermal plasma reactor, a catalytic convertor, and a sulfur condenser. The plasma reactor includes a dielectric barrier discharge, corona discharge, pulse corona discharge, glow discharge, microwave discharge, or gliding arc discharge. The plasma reactor produces syngas, from an acid gas stream, by simultaneously splitting H₂S into H₂ and sulfur and forming CO by the reverse water gas shift reaction. The catalytic convertor includes alumina or titanium catalyst. The sulfur condenser is placed downstream of the plasma reactor to recover liquid sulfur. In some implementations, the plasma reactor includes multiple stages with a sulfur condenser placed between stages.

In some implementations, the tail gas treatment unit includes a catalytic reactor to convert all sulfur containing gases to H₂S, a quenching tower to remove excess water, an amine absorber to selectively remove H₂S and recycle it back to the plasma unit, and a regenerator to regenerate amine. The partial CO₂ capture unit installed downstream of the tail gas treatment unit includes either an amine based process, a membrane based process, or a combination of both. The partial CO₂ capture unit is used to modify the ratio of H₂ to CO in the syngas to selectively favor the production of methanol and synthetic fuels.

Other implementations are also within the scope of the following claims.

What is claimed is:

1. A method for producing methanol and synthetic fuels from an acid gas stream primarily a mixture of H₂S and CO₂, the method comprising:
receiving the acid gas stream by a plasma reactor;
producing a syngas stream by simultaneously splitting H₂S into H₂ and sulfur and forming CO by reverse water gas shift reaction in the plasma reactor;
recovering sulfur as a liquid sulfur in a sulfur condenser placed downstream of the plasma reactor;
processing a remaining gas stream from the plasma reactor in a tail gas treatment unit to capture and recycle unreacted H₂S; and
adjusting a ratio of H₂ to CO in the syngas stream using a partial CO₂ removal unit to selectively produce methanol or synthetic fuels.

2. The method of claim 1, wherein receiving acid gas stream by the plasma reactor comprises applying the plasma reactor in a non-thermal mode to the acid gas stream.

3. The method of claim 2, wherein applying the plasma reactor in a non-thermal mode to the acid gas stream comprises applying at least one of a dielectric barrier discharge, a corona discharge, a pulse corona discharge, a glow discharge, microwave discharge, or a gliding arc discharge.

4. The method of claim 3, wherein the plasma reactor includes a single catalyst, bifunctional catalyst, or a physical mixture of different catalysts such as metal sulfide, supported metal sulfide, metal nitrate, supported metal nitrides, zeolite, or carbon-based catalysts.

5. The method of claim 1, wherein the plasma reactor has multiple stages with at least one sulfur condenser between the multiple stages to recover the liquid sulfur.

6. The method of claim 1, wherein the ratio of H₂ to COₓ in the syngas stream is 2-3 for producing methanol.

7. The method of claim 6, wherein the ratio of H₂ to CO in the syngas stream is 2 for producing synthetic fuels.

8. The method of claim 1, wherein the tail gas treatment unit further comprises a catalytic reactor containing hydrogenation or hydrolysis catalyst to convert sulfur-containing gases to H₂S.

9. The method of claim 8, wherein the gases leaving the tail gas treatment unit comprise a mixture of H₂S, H₂, CO, and CO₂.

10. The method of claim 9, wherein the unreacted H₂S from the mixture is recycled back to the plasma reactor.

11. The method of claim 1, wherein the partial CO₂ removal unit is placed either upstream of the plasma reactor or downstream of the tail gas treatment unit and comprises a CO₂ selective membrane or an amine based process.

12. A method for producing methanol and synthetic fuels from an acid gas stream primarily a mixture of H₂S and CO₂ comprising:
receiving the acid gas stream by a plasma unit, wherein the acid gas stream comprises primarily of H₂S and CO₂;
producing a syngas stream in the plasma unit by splitting H₂S into H₂ and sulfur and simultaneously producing CO by a reverse water gas shift reaction;
recovering unreacted H₂S in a tail gas treatment unit, wherein the recovered H₂S is recycled back to the plasma reactor; and
using a partial CO₂ removal unit placed either upstream of the plasma unit or downstream of the tail gas treatment unit, to adjust the ratio of the H₂ and CO in the syngas stream for producing methanol and synthetic fuels.

13. The method of claim 12, wherein receiving the acid gas stream by the plasma unit further comprises:
using a non-thermal plasma reactor in multiple stages, wherein the non-thermal plasma reactor comprises one of dielectric barrier discharge, corona discharge, pulse corona discharge, glow discharge, microwave discharge, or gliding arc discharge;
using a catalytic convertor to remove SO₂, wherein the catalytic convertor uses alumina or titanium catalyst; and
using at least one sulfur condenser placed between multiple stages to recover sulfur as a liquid sulfur.

14. The method of claim 13, wherein using the non-thermal plasma reactor includes a single catalyst, bifunctional catalyst, or a physical mixture of different catalysts.

15. The method of claim 12, wherein recovering unreacted H₂S in the tail gas treatment unit further comprises a catalytic reactor that includes a hydrogenation or hydrolysis catalyst to convert all sulfur containing gas to H₂S and the gases leaving the tail gas treatment unit comprise H₂S, H₂, CO, and CO₂.

16. The method of claim 12, wherein using a slip stream from the partial CO₂ removal unit can be recycled to the plasma unit to increase CO production.

17. A system for producing methanol and synthetic fuels comprising:
a plasma unit;
a tail gas treatment unit installed downstream of the plasma unit; and
a partial CO₂ capture unit installed downstream of the tail gas treatment unit.

18. The system of claim 17, wherein the plasma unit comprises:
a non-thermal plasma reactor in multiple stages to produce a syngas stream from H₂S and CO₂ gas mixture, wherein the non-thermal plasma reactor further comprises one of dielectric barrier discharge, corona discharge, pulse corona discharge, glow discharge, microwave discharge, or gliding arc discharge;
a catalytic convertor to remove SO₂, wherein the catalytic convertor uses alumina or titanium catalyst; and
a sulfur condenser placed in between stages to recover sulfur as a liquid sulfur.

19. The system of claim 17, wherein the tail gas treatment unit comprises:
a catalytic reactor that includes a hydrogenation or hydrolysis catalyst to convert all sulfur containing gases to H₂S;
a quenching tower to remove excess water, wherein the gases leaving the quenching tower include primarily a mixture of H₂S, H₂, CO, and CO₂;
an amine absorber to selectively remove H₂S from the mixture and recycle H₂S back to the plasma unit; and
a regenerator to regenerate amine.

20. The system of claim 17, wherein the partial CO₂ removal unit is used to adjust the ratio of H₂ to CO in the syngas stream, wherein the partial CO₂ removal unit comprises one of an amine based process or a membrane based process or a combination of both.
